(19) United States
(12) Patent Application Publication
(10) Pub. No.: US 2023/0146634 A1
(43) Pub. Date: May 11, 2023

(54) HEAD-MOUNTED DEVICE WITH ELECTRONIC SCENT ASSEMBLY

(71) Applicant: Lenovo (Singapore) Pte. Ltd., Singapore (SG)

(72) Inventors: Daniel Pollack, Morrisville, NC (US); Nathan Hatfield, Morrisville, NC (US)

(21) Appl. No.: 17/519,889

(22) Filed: Nov. 5, 2021

(51) Int. Cl. A61B 5/00; A61B 5/024; A61B 5/01; A61B 5/08; A41D 13/11

(52) U.S. Cl. CPC A61B 5/6803; A61B 5/024; A61B 5/01; A61B 5/0816; A61B 5/0004; A61B 5/4836; A41D 13/11; A41D 2400/36

(57) ABSTRACT

In one aspect, a mask device may include a facial covering, at least one processor, an electronic assembly accessible to the at least one processor, and storage accessible to the at least one processor. The storage may include instructions executable by the at least one processor to identify a trigger based on a biometric of a user and, based on the identification of the trigger, release a scent from the electronic assembly into a space between the facial covering and an area where the user's nose is disposed while wearing the mask device.

### FIELD

The disclosure below relates to technically inventive, non-routine solutions that are necessarily rooted in computer technology and that produce concrete technical improvements. In particular, the disclosure below relates to head-mounted devices with electronic scent assemblies.

### BACKGROUND

As recognized herein, people might sometimes endure stress and anxiety due to various circumstances. Current electronic devices are insufficient in addressing these issues, and depending on the situation the person might not be able to take other remedial measures to address their stress or anxiety either. Indeed, sometimes operation of the electronic devices themselves can be stress-inducing. As also recognized herein, a requirement to wear a mask in certain situations can be stress-inducing but leave the person with no other option but to endure the stress. There are currently no adequate solutions to the foregoing technological problem.

### SUMMARY

Accordingly, in one aspect a mask device includes a facial covering, at least one processor, an electronic assembly accessible to the at least one processor, and storage accessible to the at least one processor. The storage includes instructions executable by the at least one processor to identify a trigger based on a biometric of a user and, based on the identification of the trigger, release a scent from the electronic assembly into a space between the facial covering and an area where the user's nose is disposed while wearing the mask device.

In various example implementations, the trigger may include the user's heart rate surpassing a threshold heart rate, a body temperature of the user surpassing a threshold temperature, and/or the user's breathing rate surpassing a threshold breathing rate.

Additionally, in various example embodiments the electronic assembly may include one or more chambers into which respective scent cartridges are insertable. The scent cartridges may house respective substances having respective scents releasable from the electronic assembly based on identification of the trigger. In some examples the mask device may include the respective scent cartridges themselves. The chambers may provide for an interference fit and/or snap fit of the scent cartridges into the electronic assembly. Additionally, the scent cartridges may be interchangeable by an end-user via removal from and insertion into the chambers. In certain examples, the mask device may also include one or more vents through which the scent is able to travel from the electronic assembly into the space.

Still further, in various examples, the at least one mask device may include a mask bearing the electronic assembly and the facial covering and also may include another device that wirelessly communicates with the mask to release the scent.

In another aspect, a method includes identifying a trigger for releasing a scent via an electronic assembly engaged with a mask and then, based on identifying the trigger, actuating the electronic assembly engaged with the mask to release the scent into a space between a facial covering of the mask and an area where a user's nose is disposed while wearing the mask.

In some examples, the method may therefore include identifying the trigger based on a biometric of the user, such as a biometric related to a current heart rate of the user and/or a current body temperature of the user. However, the method may additionally or alternatively include identifying the trigger based on execution of a video game, based on presentation of an audio video (A/V) program such as a movie or a television show, and/or based on presentation of music or other audio content.

In still another aspect, a scent cartridge receptacle includes a housing engageable with a face mask and/or other head-mountable device, one or more chambers in the housing into which respective scent cartridges are receivable, and electronic circuitry for communicating with at least one processor to release scent from one or more of the respective scent cartridges via the mask and/or other head-mountable device.

In some examples, the scent cartridge receptacle may even include the at least one processor itself.

The details of present principles, both as to their structure and operation, can best be understood in reference to the accompanying drawings, in which like reference numerals refer to like parts, and in which:

### BRIEF DESCRIPTION OF THE DRAWINGS

FIG. 1 is a block diagram of an example system consistent with present principles;

FIG. 2 is a block diagram of an example network of devices consistent with present principles;

FIG. 3 shows a front perspective view of an example smart mask consistent with present principles;

FIG. 4 shows a rear perspective view of an example smart mask consistent with present principles;

FIG. 5 shows an example front perspective view of an example smart mask consistent with present principles but while worn by an end-user;

FIG. 6 shows an example scent cartridge that may be used consistent with present principles;

FIG. 7 illustrates an example wearable device being worn on a user's wrist to sense one or more biometrics of the user consistent with present principles;

FIG. 8 illustrates paired Bluetooth communication that may occur between devices to undertake present principles;

FIG. 9 shows an example status graphical user interface (GUI) indicating the connection status of various devices that may be used to undertake present principles;

FIG. 10 illustrates example logic in example flow chart format that may be executed by a device consistent with present principles;

FIG. 11 shows an example GUI through which a user may specify various scent cartridges that have been inserted into a smart mask consistent with present principles;

FIG. 12 shows an example GUI that may be used to command the smart mask to release a scent consistent with present principles;

FIG. 13 shows an example GUI that may be presented responsive to scent for a particular cartridge running out consistent with present principles; and

FIG. 14 shows an example settings GUI that may be used to configure one or more settings of a device or application to execute for scent release consistent with present principles.

### DETAILED DESCRIPTION

Among other things, the detailed description below relates to use of a smart mask to combat anxiety and stress by subtly releasing scents into the user's vicinity. The mask or connected device may measure the amount of stress in the body by regulation and release a soothing scent depending upon the user's choice to help them relax. Additionally or alternatively, the mask may be programmed to further immerse a user into a relaxation state by enhancing olfactory senses. Other applications may include enhancing immersion with event-based dispersions within a videogame (e.g., wood or fire scent) or the release of peppermint scents to relax users.

In order to monitor a user's stress level, an additional sensory wristband may be utilized in certain implementations to monitor the heartrate of an individual. When reaching specific heartrates, the wrist sensor may then send a signal to the mask via Bluetooth. The mask may then act accordingly by subtly releasing a scent to the user to help calm him/her. Scent dispersion can be autonomous based on biosignature data captured from other wearables as well (e.g., once heart or respiration rate returns to baseline, scent dispersion may taper off and/or end).

Additionally, different scents may be provided and programmed accordingly depending on user preference. Scents can be stored in small cartridges that can be inserted and removed from the smart mask as they run out. This can also be set in a software app that the user can download and configure. Lavender, jasmine, lemon, and other scents may thus be used to combat stress and bring a calm state of mind.

Additionally or alternatively, as mentioned above the smart mask may be used to enhance a user's experience with electronic entertainment such as movies, videogames, and music. Scents can be configured to release at specific times according to the app to enrich the user's experience and further immerse the user into their entertainment.

Prior to delving further into the details of the instant techniques, note with respect to any computer systems discussed herein that a system may include server and client components, connected over a network such that data may be exchanged between the client and server components. The client components may include one or more computing devices including televisions (e.g., smart TVs, Internet-enabled TVs), computers such as desktops, laptops and tablet computers, so-called convertible devices (e.g., having a tablet configuration and laptop configuration), and other mobile devices including smart phones. These client devices may employ, as non-limiting examples, operating systems from Apple Inc. of Cupertino Calif., Google Inc. of Mountain View, Calif., or Microsoft Corp. of Redmond, Wash. A Unix® or similar such as Linux® operating system may be used. These operating systems can execute one or more browsers such as a browser made by Microsoft or Google or Mozilla or another browser program that can access web pages and applications hosted by Internet servers over a network such as the Internet, a local intranet, or a virtual private network.

As used herein, instructions refer to computer-implemented steps for processing information in the system. Instructions can be implemented in software, firmware or hardware, or combinations thereof and include any type of programmed step undertaken by components of the system; hence, illustrative components, blocks, modules, circuits, and steps are sometimes set forth in terms of their functionality.

A processor may be any single- or multi-chip processor that can execute logic by means of various lines such as address lines, data lines, and control lines and registers and shift registers. Moreover, any logical blocks, modules, and circuits described herein can be implemented or performed with a system processor, a digital signal processor (DSP), a field programmable gate array (FPGA) or other programmable logic device such as an application specific integrated circuit (ASIC), discrete gate or transistor logic, discrete hardware components, or any combination thereof designed to perform the functions described herein. A processor can also be implemented by a controller or state machine or a combination of computing devices. Thus, the methods herein may be implemented as software instructions executed by a processor, suitably configured application specific integrated circuits (ASIC) or field programmable gate array (FPGA) modules, or any other convenient manner as would be appreciated by those skilled in those art. Where employed, the software instructions may also be embodied in a non-transitory device that is being vended and/or provided that is not a transitory, propagating signal and/or a signal per se (such as a hard disk drive, CD ROM, or Flash drive). The software code instructions may also be downloaded over the Internet. Accordingly, it is to be understood that although a software application for undertaking present principles may be vended with a device such as the system 100 described below, such an application may also be downloaded from a server to a device over a network such as the Internet.

Software modules and/or applications described by way of flow charts and/or user interfaces herein can include various sub-routines, procedures, etc. Without limiting the disclosure, logic stated to be executed by a particular module can be redistributed to other software modules and/or combined together in a single module and/or made available in a shareable library. Also, the user interfaces (UI)/graphical UIs described herein may be consolidated and/or expanded, and UI elements may be mixed and matched between UIs.

Logic when implemented in software, can be written in an appropriate language such as but not limited to hypertext markup language (HTML)-5, Java/JavaScript, C # or C++, and can be stored on or transmitted from a computer-readable storage medium such as a random access memory (RAM), read-only memory (ROM), electrically erasable programmable read-only memory (EEPROM), a hard disk drive or solid state drive, compact disk read-only memory (CD-ROM) or other optical disk storage such as digital versatile disc (DVD), magnetic disk storage or other magnetic storage devices including removable thumb drives, etc.

In an example, a processor can access information over its input lines from data storage, such as the computer readable storage medium, and/or the processor can access information wirelessly from an Internet server by activating a wireless transceiver to send and receive data. Data typically is converted from analog signals to digital by circuitry between the antenna and the registers of the processor when being received and from digital to analog when being transmitted. The processor then processes the data through its shift registers to output calculated data on output lines, for presentation of the calculated data on the device.

Components included in one embodiment can be used in other embodiments in any appropriate combination. For example, any of the various components described herein and/or depicted in the Figures may be combined, interchanged, or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

The term “circuit” or “circuitry” may be used in the summary, description, and/or claims. As is well known in the art, the term “circuitry” includes all levels of available integration, e.g., from discrete logic circuits to the highest level of circuit integration such as VLSI, and includes programmable logic components programmed to perform the functions of an embodiment as well as general-purpose or special-purpose processors programmed with instructions to perform those functions.

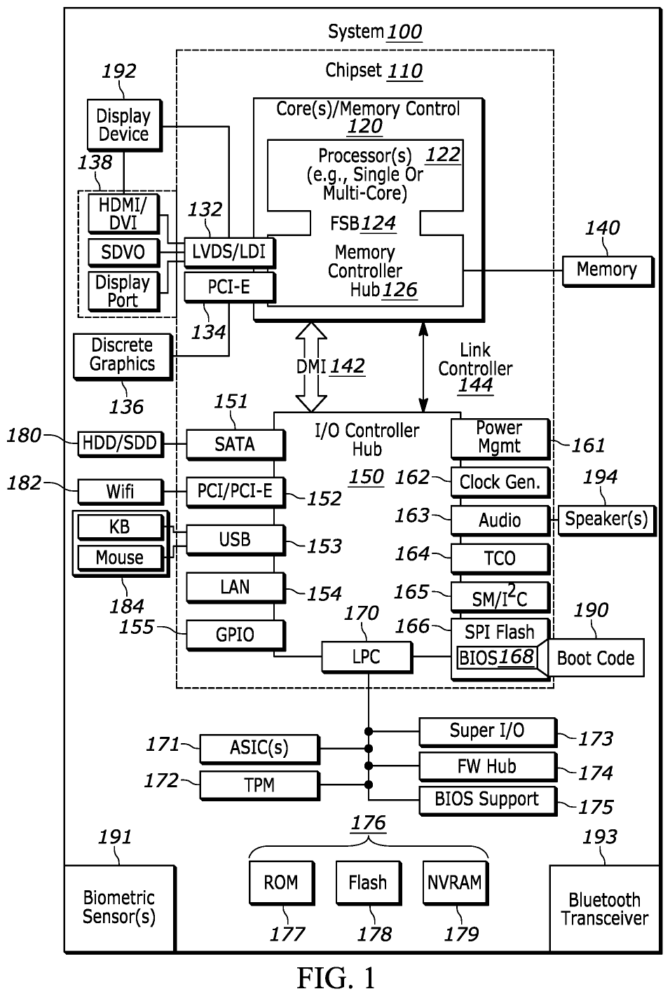

Now specifically in reference to FIG. 1, an example block diagram of an information handling system and/or computer system 100 is shown that is understood to have a housing for the components described below. Note that in some embodiments the system 100 may be a desktop computer system, such as one of the ThinkCentre® or ThinkPad® series of personal computers sold by Lenovo (US) Inc. of Morrisville, N.C., or a workstation computer, such as the ThinkStation®, which are sold by Lenovo (US) Inc. of Morrisville, N.C.; however, as apparent from the description herein, a client device, a server or other machine in accordance with present principles may include other features or only some of the features of the system 100. Also, the system 100 may be, e.g., a game console such as XBOX®, and/or the system 100 may include a mobile communication device such as a mobile telephone, notebook computer, and/or other portable computerized device.

As shown in FIG. 1, the system 100 may include a so-called chipset 110. A chipset refers to a group of integrated circuits, or chips, that are designed to work together. Chipsets are usually marketed as a single product (e.g., consider chipsets marketed under the brands INTEL®, AMD®, etc.).

In the example of FIG. 1, the chipset 110 has a particular architecture, which may vary to some extent depending on brand or manufacturer. The architecture of the chipset 110 includes a core and memory control group 120 and an I/O controller hub 150 that exchange information (e.g., data, signals, commands, etc.) via, for example, a direct management interface or direct media interface (DMI) 142 or a link controller 144. In the example of FIG. 1, the DMI 142 is a chip-to-chip interface (sometimes referred to as being a link between a “northbridge” and a “southbridge”).

The core and memory control group 120 include one or more processors 122 (e.g., single core or multi-core, etc.) and a memory controller hub 126 that exchange information via a front side bus (FSB) 124. As described herein, various components of the core and memory control group 120 may be integrated onto a single processor die, for example, to make a chip that supplants the “northbridge” style architecture.

The memory controller hub 126 interfaces with memory 140. For example, the memory controller hub 126 may provide support for DDR SDRAM memory (e.g., DDR, DDR2, DDR3, etc.). In general, the memory 140 is a type of random-access memory (RAM). It is often referred to as “system memory.”

The memory controller hub 126 can further include a low-voltage differential signaling interface (LVDS) 132. The LVDS 132 may be a so-called LVDS Display Interface (LDI) for support of a display device 192 (e.g., a CRT, a flat panel, a projector, a touch-enabled light emitting diode (LED) display or other video display, etc.). A block 138 includes some examples of technologies that may be supported via the LVDS interface 132 (e.g., serial digital video, HDMI/DVI, display port). The memory controller hub 126 also includes one or more PCI-express interfaces (PCI-E) 134, for example, for support of discrete graphics 136. Discrete graphics using a PCI-E interface has become an alternative approach to an accelerated graphics port (AGP). For example, the memory controller hub 126 may include a 16-lane (x16) PCI-E port for an external PCI-E-based graphics card (including, e.g., one of more GPUs). An example system may include AGP or PCI-E for support of graphics.

In examples in which it is used, the I/O hub controller 150 can include a variety of interfaces. The example of FIG. 1 includes a SATA interface 151, one or more PCI-E interfaces 152 (optionally one or more legacy PCI interfaces), one or more universal serial bus (USB) interfaces 153, a local area network (LAN) interface 154 (more generally a network interface for communication over at least one network such as the Internet, a WAN, a LAN, a Bluetooth network using Bluetooth 5.0 communication, etc. under direction of the processor(s) 122), a general purpose I/O interface (GPIO) 155, a low-pin count (LPC) interface 170, a power management interface 161, a clock generator interface 162, an audio interface 163 (e.g., for speakers 194 to output audio), a total cost of operation (TCO) interface 164, a system management bus interface (e.g., a multi-master serial computer bus interface) 165, and a serial peripheral flash memory/controller interface (SPI Flash) 166, which, in the example of FIG. 1, includes basic input/output system (BIOS) 168 and boot code 190. With respect to network connections, the I/O hub controller 150 may include integrated gigabit Ethernet controller lines multiplexed with a PCI-E interface port. Other network features may operate independent of a PCI-E interface.

The interfaces of the I/O hub controller 150 may provide for communication with various devices, networks, etc. For example, where used, the SATA interface 151 provides for reading, writing, or reading and writing information on one or more drives 180 such as HDDs, SDDs or a combination thereof, but in any case, the drives 180 are understood to be, e.g., tangible computer readable storage mediums that are not transitory, propagating signals. The I/O hub controller 150 may also include an advanced host controller interface (AHCI) to support one or more drives 180. The PCI-E interface 152 allows for wireless connections 182 to devices, networks, etc. The USB interface 153 provides for input devices 184 such as keyboards (KB), mice and various other devices (e.g., cameras, phones, storage, media players, etc.).

In the example of FIG. 1, the LPC interface 170 provides for use of one or more ASICs 171, a trusted platform module (TPM) 172, a super I/O 173, a firmware hub 174, BIOS support 175 as well as various types of memory 176 such as ROM 177, Flash 178, and non-volatile RAM (NVRAM) 179. With respect to the TPM 172, this module may be in the form of a chip that can be used to authenticate software and hardware devices. For example, a TPM may be capable of performing platform authentication and may be used to verify that a system seeking access is the expected system.

The system 100, upon power on, may be configured to execute boot code 190 for the BIOS 168, as stored within the SPI Flash 166, and thereafter processes data under the control of one or more operating systems and application software (e.g., stored in system memory 140). An operating system may be stored in any of a variety of locations and accessed, for example, according to instructions of the BIOS 168.

As also shown in FIG. 1, the system 100 may include one or more biometric sensors 191 for sensing biometrics of a user consistent with present principles. Thus, the sensors 191 may include, for example, a pulse/heart rate sensor, a blood pressure sensor, a perspiration sensor (e.g., liquid sensor, capacitance sensor, etc.), a body or skin temperature sensor, a lung input/output sensor or other breath sensor (such as a microphone picking up on audible breaths or an atmospheric pressure sensor, motion sensor, or piezoresistive breath sensor), etc. Other examples of biometric sensors include a blood oxygen sensor, a glucose and/or blood sugar sensor, a brain activity sensor, and a retina and/or iris sensor.

Still further, the system 100 may include a Bluetooth transceiver and/or other short-range wireless communication interface 193 for use to communicate with paired wearable devices, smart phones, etc. consistent with present principles. For example, one or more publicly-available Bluetooth specifications may be used for Bluetooth communication using the transceiver 193. Thus, the Bluetooth transceiver 193 may be a classic Bluetooth transceiver and/or a Bluetooth low energy (BLE) transceiver (e.g., Bluetooth 5.0 transceiver) for communicating with other devices using Bluetooth communication protocols. Additionally, as alluded to above the transceiver 193 may also be configured for communicating using other wireless protocols and may therefore establish a Zigbee transceiver, Z-wave transceiver, near field communication (NFC) transceiver, infrared transceiver, a Wi-Fi direct transceiver, and/or wireless universal serial bus (USB) transceiver, for example.

Additionally, though not shown for simplicity, in some embodiments the system 100 may include a gyroscope that senses and/or measures the orientation of the system 100 and provides related input to the processor 122, as well as an accelerometer that senses acceleration and/or movement of the system 100 and provides related input to the processor 122. Still further, the system 100 may include an audio receiver/microphone that provides input from the microphone to the processor 122 based on audio that is detected, such as via a user providing audible input to the microphone. The system 100 may also include a camera that gathers one or more images and provides the images and related input to the processor 122. The camera may be a thermal imaging camera, an infrared (IR) camera, a digital camera such as a webcam, a three-dimensional (3D) camera, and/or a camera otherwise integrated into the system 100 and controllable by the processor 122 to gather still images and/or video. Also, the system 100 may include a global positioning system (GPS) transceiver that is configured to communicate with at least one satellite to receive/identify geographic position information and provide the geographic position information to the processor 122. However, it is to be understood that another suitable position receiver other than a GPS receiver may be used in accordance with present principles to determine the location of the system 100.

It is to be understood that an example client device or other machine/computer may include fewer or more features than shown on the system 100 of FIG. 1. In any case, it is to be understood at least based on the foregoing that the system 100 is configured to undertake present principles.

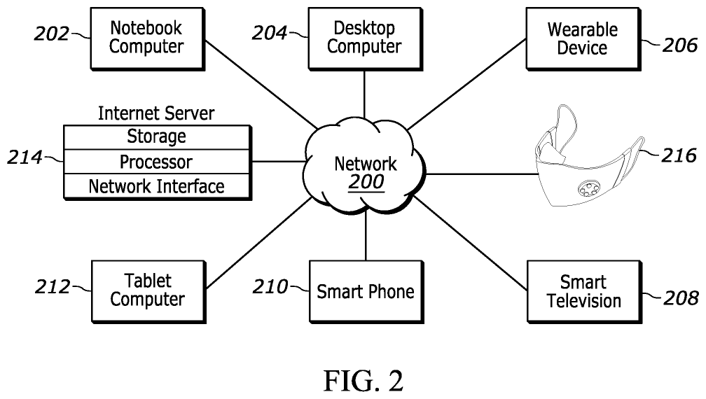

Turning now to FIG. 2, example devices are shown communicating over a network 200 such as the Internet or a Bluetooth network in accordance with present principles. It is to be understood that each of the devices described in reference to FIG. 2 may include at least some of the features, components, and/or elements of the system 100 described above. Indeed, any of the devices disclosed herein may include at least some of the features, components, and/or elements of the system 100 described above.

FIG. 2 shows a notebook computer and/or convertible computer 202, a desktop computer 204, a wearable device 206 such as a smart watch, a smart television (TV) 208, a smart phone 210, a tablet computer 212, an aroma therapy or smart mask 216, and a server 214 such as an Internet server that may provide cloud storage accessible to the devices 202-212, 216. It is to be understood that the devices 202-216 may be configured to communicate with each other over the network 200 to undertake present principles.

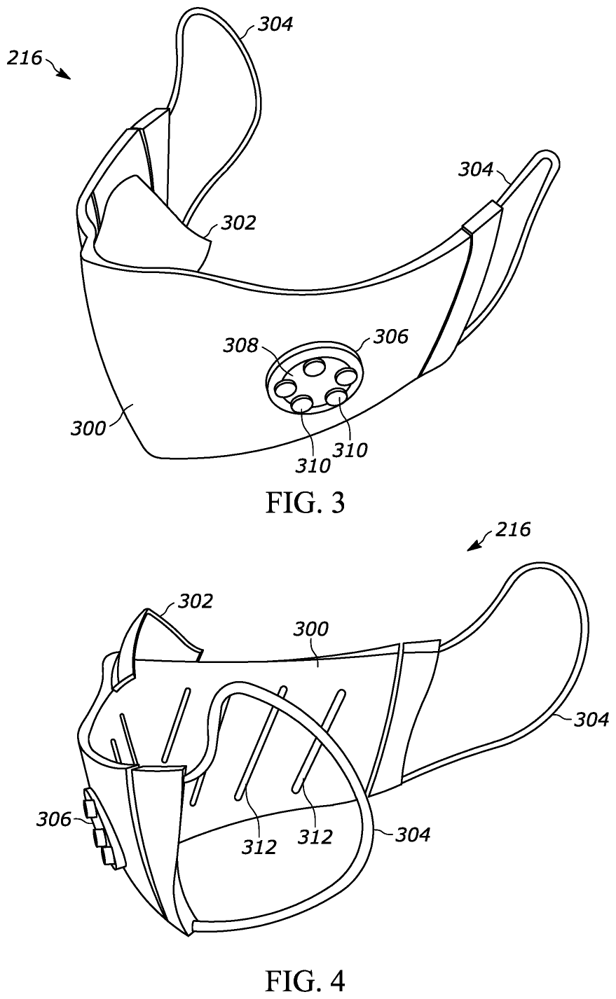

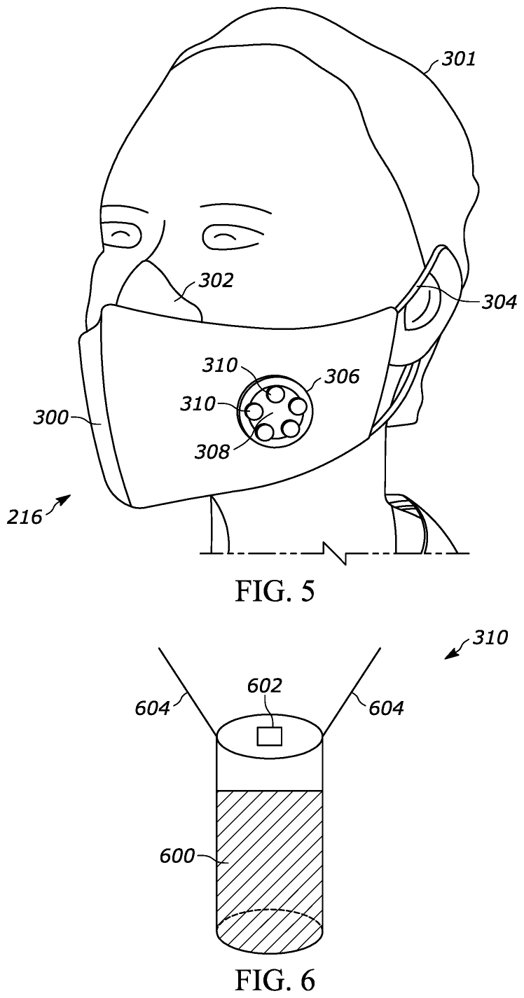

Continuing the detailed description in cross-reference to FIGS. 3-5, they show the example smart mask 216 in more detail. FIG. 3 shows a front perspective view of the mask 216, FIG. 4 shows a rear perspective view of the mask 216, and FIG. 5 shows a front perspective view of the mask 216 while being worn by an end-user/wearer 301.

As shown in these figures, the mask 216 may include a rigid or non-rigid facial covering 300 that may be made of cloth, mesh, and/or other suitable material. The facial covering 300 may cover all exterior portions of the wearer's mouth when worn correctly and may, in some examples, include a discrete a nose piece 302 to cover the wearer's nose from the bottom of the nostrils all the way up to the top of the bridge. Additionally, the mask 216 may include ear straps 304 at respective mask side portions to extend over the wearer's ears to hold the mask 216 in place on the person's face.

As also shown in FIGS. 3-5, the mask 216 may include an electronic assembly 306 that may have and/or at least be accessible to a processor and/or electronic storage (not shown). The assembly 306 may also include other electronic circuitry for use consistent with present principles. For example, the assembly 306 may include a scent cartridge receptacle 308 that may include a housing engageable with the mask 216 (and/or other head-mountable device). The housing may be made of plastic, wood, composite materials, one or more metals, and/or other suitable materials. The housing may establish one or more hollow chambers into which respective scent cartridges 310 can be received as inserted by an end-user. For example, the chambers may provide for an interference fit and/or snap fit of the scent cartridges 310 into respective chambers in the housing of the receptacle 308 so that they may interchangeable via an end-user sliding them in and/or snapping/locking them into place and later easily removing them from the chambers by pressing a spring-loaded depressable release button(s) or actuating one or more release tab(s). Thus, the user need not use a specialized tool or any other object for that matter to engage and disengage the cartridges 310 from the receptacle 308 (e.g., other than the user's fingers).

As for the cartridges 310 themselves, they may be cylindrically-shaped tubes if desired, or may be cylindrically-shaped disks in particular to have a low profile/height to remain unobtrusive to the user's touch and sight while disposed within the receptable 308. However, the cartridges 310 may be in other shapes as well depending on desired implementation. For example, the cartridges may be cube-shaped or tetrahedron-shaped. But regardless, further note that each cartridge 310 and hence each corresponding chamber in the receptacle 308 need not be the same size so that, for example, a larger cartridge for a more-frequently used scent may be placed into a corresponding larger chamber for closely-receiving that respective cartridge via a snap or interference fit, while a smaller cartridge for a less-frequently used scent may be placed into a smaller corresponding chamber.

Note that the electronic circuitry of the assembly 306 may further include other components not shown for simplicity, such as a Bluetooth transceiver for communicating with at least one processor on one or more other devices to release scent from one or more of the respective scent cartridges 310 via the mask as described herein. The electronic circuitry may also include motors, linkage, gears, etc. for the processor on the mask 216 or other device to electronically control the assembly 306 and scent cartridge receptacle 308 in particular to release scent from one or more of the cartridges 310 in the receptacle 308. RAM, rechargeable batteries, and other system components such as any of those described above in reference to the system 100 may also be included as part of the circuitry of the mask 216.

Additionally, as best shown in FIG. 4, the facial covering may have not just an outer vertical sheet for covering the person's face but in some examples may also have an inner vertical sheet. The inner sheet may include one or more vents 312 through which scent from one or more of the cartridges 310 is able to travel from the electronic assembly 306 (which may be sown, glued, or otherwise integrated into the outer sheet) into a space between the facial covering 300 and an area inside the mask 216 where the user's nose is disposed while wearing the mask so that the user can smell the scent upon the scent traveling from the assembly, between the two sheets of the facial covering 300, and ultimately to the user's nostrils. However, further note that while the two sheets may be sown together or otherwise attached to each other, they may form a single unitary structure of molded rigid or non-rigid material or other material with hollow areas inside for the scent to travel from the assembly 306 to the user's nose. Other configurations are also possible.

As for the scent itself, note that it may be a gas or other airborne substance such as a mist having liquid droplets, for example. While housed in a respective cartridge 310, the scent may be reduced to or embodied in a solid (e.g., gel) or liquid that emits smell when exposed to the air through a duct or other component on the cartridge that mechanically/electrically opens and closes under control of the linkage in the assembly 306/receptable 308. However, the scent may also be embodied as a compressed gas within the cartridge 310 with the pressure forcing exertion of the gas out of the duct when the duct is opened. A compressed liquid may also be used to provide the aforementioned mist, for example. But regardless, note that the duct on each cartridge 310 may be in fluid communication with the vents 312 when engaged with the mask 216 to deliver the scent to the nostrils of the user 301.

Further note that the receptacle 308 itself may be integral with the rest of the assembly 306, or may be discrete and separate but snap into the assembly 306 via an appropriately-shaped (e.g., circular-shaped) and electrically-connected grommet forming part of the assembly 306.

Now in reference to FIG. 6, an example cylindrical scent cartridge 310 is shown in greater detail. As shown, the cartridge 310 may house a substance 600 such as solid, liquid, or pressurized gas as described above and may also include a duct 602 as described above. Though not shown for simplicity, a mechanically and/or electrically-slidable door may cover and uncover the duct 602 under control of a processor to selectively release scent consistent with present principles, though a plug or door on the receptacle 308 itself may also be used to cover and uncover the duct 602 while the cartridge 310 is engaged with the receptacle 308, if desired. The cartridge 310 may also include one or more tabs 604 that can be pinched together while the cartridge 310 is snapped or otherwise locked into place within the receptacle 308 to release the cartridge 310 and pull it out from the receptacle 308. However, a spring-loaded button may also be used to release the cartridge 310 from the receptacle 308, as may other suitable mechanisms.

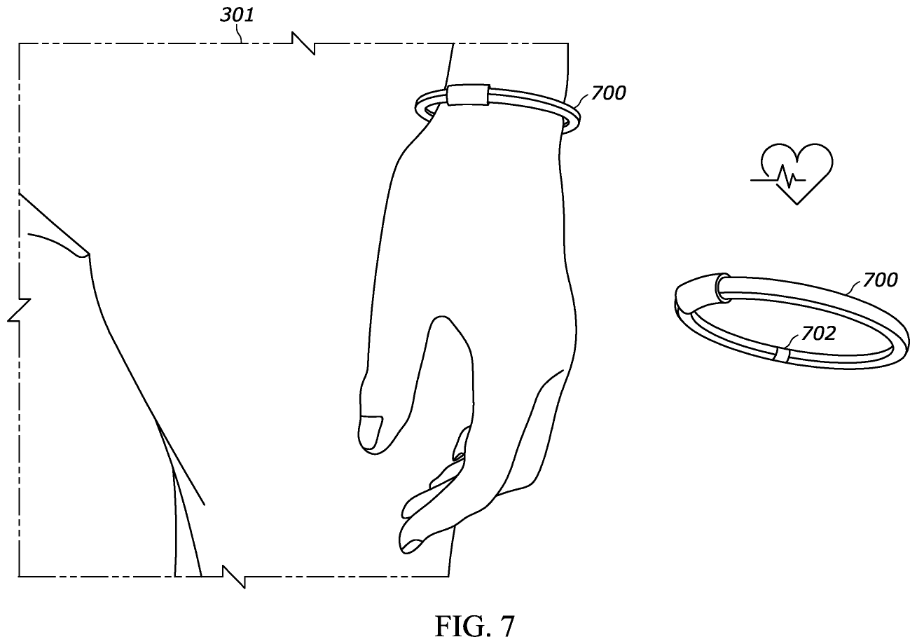

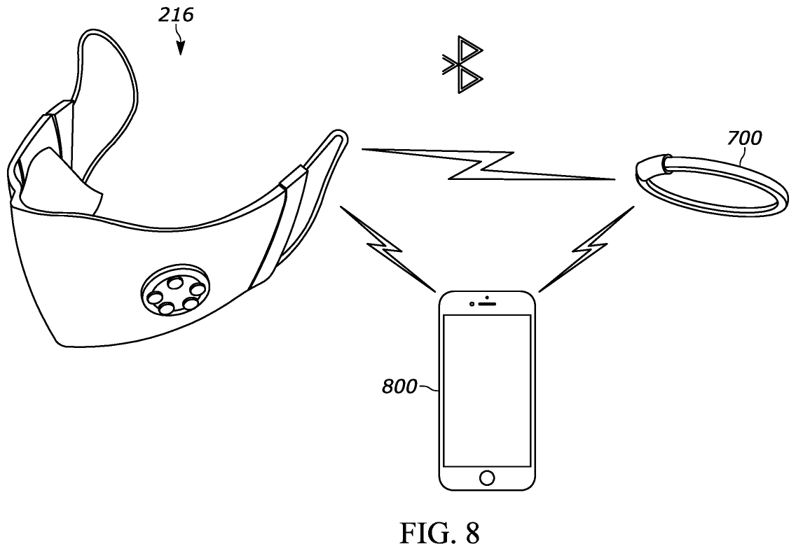

Continuing the detailed description in reference to FIG. 7, it illustrates that the user 301 may don a wearable device 700 that may have one or more biometric sensors 702 on an inside surface to contact the user's skin while the wearable device 700 is worn. For example, the biometric sensor 702 may be a temperature sensor, optical heart rate sensor, electrode hear rate sensor, or other biometric sensor consistent with present principles. The device 700 is illustrated in FIG. 7 as an electronic bracelet, but note that in other examples the device may be a smart watch, electronic skin patch, or any other suitable wearable device that can measure user biometrics consistent with present principles. The device might also be an implantable device, as another example. But regardless of device type, further note that the device may have system components as described above in reference to FIG. 1, including a processor for receiving input from the biometric sensor 702 itself as well as a Bluetooth or other wireless transceiver for communicating with other devices including the mask 216 and a smart phone 800 as shown in FIG. 8.

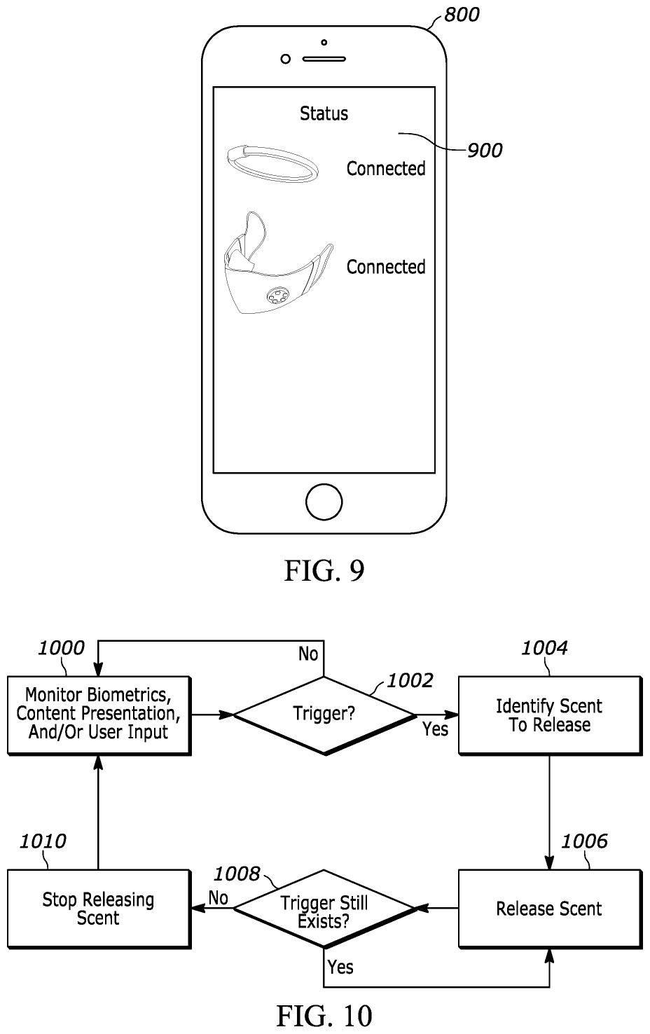

FIG. 9 further illustrates. The smart phone 800 is shown while presenting a connection status graphical user interface (GUI) 900 on its display. The GUI 900 includes images and text as shown that indicate that both the wearable device 700 and mask 216 are paired with or otherwise currently connected to the phone 800 via wireless communication consistent with present principles. Thus, in one example implementation the smart phone 800 may receive biometric input from one or more biometric sensors on the wearable device 700 via Bluetooth communication, determine whether to release scent via the mask 216, and then communicate via Bluetooth with the mask 216 itself to control the mask 216 to release the scent.

FIG. 10 illustrates even further via example logic that may be executed by a device such as the smart phone 800 consistent with present principles. However, the logic may also be executed by another type of device such as laptop computer, tablet computer, or any other suitable device being used. The logic might also be executed by the wearable device 700 itself in conjunction with the smart phone 800 or even a remotely-located server in any appropriate combination, or the wearable device 700 may even execute the logic by itself in a two-device implementation where functions otherwise executed by the smart phone 800 are executed by the wearable device 700 as communicating directly with the mask 216 to release scent (e.g., no smart phone is present and instead bi-directional communication is used rather than the three-way communication shown in FIG. 8). As yet another example implementation, the mask 216 or other head-mounted device that is being worn may include its own biometric sensors and perform one or all steps of the logic of FIG. 10 without communicating with a wearable device, smart phone, etc. In any case, further note that while the logic of FIG. 10 is shown in flow chart format, other suitable logic may also be used.

Now describing the logic itself in more detail, it may begin at block 1000 where the device may monitor one or more biometrics of a user based on input from one or more biometric sensors as described herein. For example, at block 1000 the device may monitor the user's heart rate, body temperature, and/or breathing rate. Additionally or alternatively, at block 1000 the device may monitor the presentation of content as presented at the device undertaking the logic of FIG. 10 or another connected/paired device such as a television, video game console, and/or smartphone to eventually release scent at a designated time within presentation of the content. The content may be established by a video game, an audio video (A/V) program such as a movie or television show or streaming Internet video, audio-only music, or other audio content (such as a podcast or other voice recording), or other type of content.

From block 1000 the logic may then proceed to decision diamond 1002 where the device may identify whether a trigger exists for scent release. The trigger may be identified based on a biometric of a user passing a threshold for the respective biometric. For example, the trigger may be the user's current heart rate surpassing a threshold heart rate as sensed via a heart rate sensor, a current body temperature of the user surpassing a threshold temperature as sensed via a temperature sensor, and/or the user's current breathing rate surpassing a threshold breathing rate as sensed by a breath sensor, any, or all of which might indicate the user undergoing stress that electronic scent release might help alleviate.

In addition to or in lieu of the foregoing but also at diamond 1002, the logic may monitor for other triggers such as a designated time within presentation of any of the content described above being reached. The designated time may be indicated in and/or preprogrammed into the digital file for the content itself so that the device may recognize the time point within the content's playtime or recognize a predefined occurrence within the content as being reached and then release a designated scent indicated in the digital file. For example, a lemon scent may be programmed for release at the 1:25:02 hour/minute/second mark for a movie, or a smoke/wood scent may be programmed for release whenever a user comes across a virtual fireplace as part of playing a video game.

A negative determination may cause the logic to revert back to block 1000 and proceed again therefrom. However, responsive to an affirmative determination at diamond 1002 that a trigger has been identified, the logic may proceed to block 1004. At block 1004 the device may identify a particular scent to release (e.g., based on user preference, based on device or application programming, based on a scent specified in the digital file of the content, etc.). The logic may then proceed to block 1006 where the device may actuate an electronic assembly on the mask (e.g., the assembly 306) to release the identified scent via the electronic assembly consistent with the disclosure above. For example, if the device executing the logic of FIG. 10 is a smart phone, the smart phone may issue a command over Bluetooth to the mask to release the scent. Or if the device executing the logic of FIG. 10 is the mask itself, the mask may itself control the circuitry of its electronic assembly to release the scent.

From block 1006 the logic may then proceed to decision diamond 1008. At diamond 1008 the logic may determine if the trigger still exists. For example, at diamond 1008 the logic may determine whether a given biometric is still above the corresponding threshold or whether the point in the content's presentation at which the scent should be released or continue to be released is still ongoing. An affirmative determination at diamond 1008 may cause the logic to revert back to block 1006 to continue releasing the identified scent.

However, if the point in the content has passed where scent is to be released or the user's biometric goes back below the corresponding threshold, a negative determination may be made instead and the logic may therefore proceed to block 1010 where the device may control the electronic assembly on the mask to stop releasing the scent identified at block 1004 (e.g., stop releasing scent altogether or begin releasing another scent as appropriate). From block 1010 the logic may revert back to block 1000 and proceed therefrom.

Further describing example triggers that might trigger the device(s) of FIG. 10 to release scent, in some examples the trigger may be established by a user command to release a particular scent. The command may be received via selection of a depressable or touch-enabled button on the mask itself (or connected wearable device) to release a designated scent where, for example, there are multiple buttons on the mask (or separate wearable) that are each associated with a different respective chamber/cartridge for the user to thus be able to choose which scent to release. For example, each cartridge on the mask may itself be depressable inward toward the user's face under spring bias to establish a button for scent release. Additionally or alternatively, the button may be presented as part of a GUI presented on the display of the wearable device or connected smartphone, as will be described later.

Also note that a trigger for scent release might be a biometric of the user going below a certain biometric threshold rather than above it. For example, in addition to or in lieu of releasing a lavender, jasmine, lemon, pine, or peppermint scent for relaxation if a user's heart rate goes above a first threshold heart rate, if the user's heart rate goes below a different low heart rate threshold, then another type of scent may be released such as a scent generated by smelling salts in one of the mask's cartridges. The smelling salts may be established by ammonia inhalants such as a combination of ammonium carbonate and perfume, for example. Thus, if the user's heart rate drops past the lower threshold due to the user falling asleep, fainting, or otherwise losing consciousness, the smelling salts might help wake the user up.

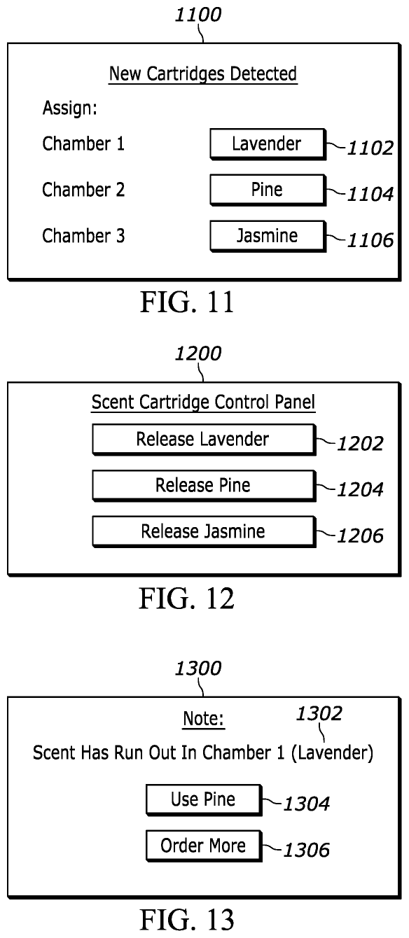

Continuing the detailed description in reference to FIG. 11, suppose a user inserts one or more new scent cartridges into a scent cartridge receptacle as described above. The circuitry within the mask may detect as much, e.g. using optical presence sensors or a detected electrical current change based on the cartridge(s) being inserted to contact or complete an electrical path within the circuitry. Responsive to detecting scent cartridge insertion, the GUI 1100 may be presented on a display such as the display of the wearable device or smart phone in communication with the mask.

As shown in FIG. 11, the GUI 1100 allows a user to indicate to the device(s) which scent cartridges have which scents for the device to then control scent release from the corresponding chamber into which the cartridge has been inserted. The chambers themselves may be visually numbered on the mask itself, for example, so that the user would know which scent cartridge is being inserted into which chamber. The user may then select a corresponding selector 1102-1106 for each respective chamber which in turn may cause a drop-down menu or other GUI element to be presented from which the user can select a particular scent to be associated with that respective chamber. However, notwithstanding FIG. 11, further note that in some examples the smart mask or connected device may auto-detect scents as well based on various configurations of the cartridges themselves if desired (e.g., passive RFID communication using a passive RFID tag on the cartridge and active RFID transceiver on the mask's electronic assembly).

FIG. 12 then shows that a GUI 1200 may be presented on the display for a user to provide a command to release a particular scent at a time of the user's choosing after the user has indicated which scent cartridges are engaged with which chambers, or after the device auto-detects as much. For example, the GUI 1200 (and the other GUIs described herein for that matter) may be presented as part of an application (“app”) executing at the user's smart phone to control the electronic assembly of the mask. Thus, the GUI 1200 may be invoked at the user's choosing by launching the app, and either having the GUI 1200 automatically presented or navigating to it within the app itself.

As shown in FIG. 12, the GUI 1200 may establish a scent cartridge control panel where the user may provide a command to release a designated scent associated with a respective selector/button 1202-1206 by selecting the associated button via touch or cursor input for example. In the example shown, the scents are lavender, pine, and jasmine. The selected scent may continue to be released until the respective selector 1202-1206 is selected again, and/or the scent may be released for a threshold time responsive to the initial selection of the selector 1202-1206 and the mask may then stop releasing the scent after expiration of the threshold time. The threshold time might be five seconds, for example.

FIG. 13 illustrates that responsive to the mask or other device detecting that a given scent cartridge is empty (e.g., using an infrared proximity sensor or other optical sensor if the cartridge's housing is transparent), a GUI 1300 may be presented. As shown in FIG. 13, the GUI 1300 may include an indication 1302 that lavender scent has run out or is otherwise no longer available via the corresponding cartridge as disposed in a certain chamber (“chamber 1” in this example). The indication 1302 might also specify the particular scent associated with that cartridge as also shown. The user may then elect to select selector 1304 to use pine scent from another cartridge that is currently engaged with the mask in instances where the depleted lavender scent would otherwise be released. Additionally or alternatively, the user may select the selector 1306 to launch an online portal through which more lavender or other cartridges may be ordered.

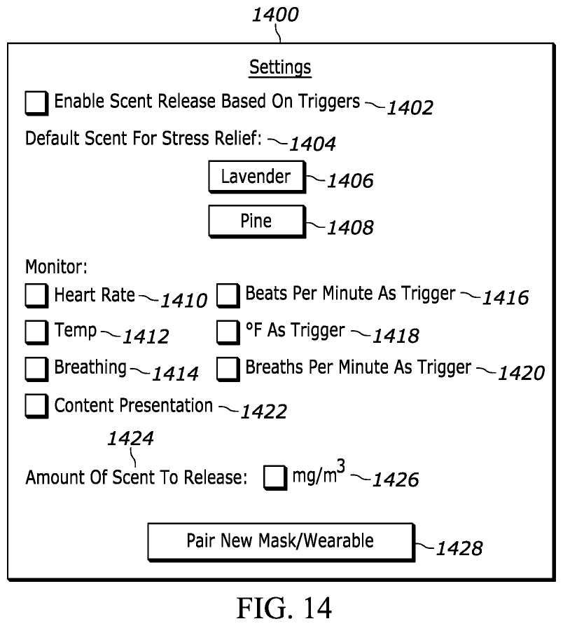

Continuing the detailed description in reference to FIG. 14, it shows an example settings GUI 1400 that may be presented on a display based on a user navigating the app described above so that the user may configure one or more settings of the device/mask/app to execute consistent with present principles. Note that in the present example, each of the options to be discussed below may be selected by directing touch or cursor input to the respective check box adjacent to the respective option.

As shown in FIG. 14, the GUI 1400 may include a first option that may be selectable to set or configure the device/mask/app to undertake present principles. For example, the option 1402 may be selected to enable the mask to, for multiple instances in the future, perform the scent-release functions described above in reference to FIGS. 3-9, execute the logic of FIG. 10, etc.

The GUI 1400 may also include a setting 1404 at which a user can specify a particular scent to use for stress relief, where stress might be detected based on a certain biometric like current heart rate going above a heart rate threshold. Thus, the user may select the selector 1406 to select lavender as the associated scent or may select the selector 1408 to select pine as the associated scent. However, other available scents may be listed and presented if available.

The GUI 1400 may also include respective options 1410-1414 to select respective particular biometrics for which the device/mask/app is to monitor for scent release consistent with present principles. As shown in FIG. 14, the option 1410 may be selected to use heart rate as the biometric and may even be accompanied by an input box 1416 at which the end-user himself or herself can enter numerical input to establish the threshold/trigger for scent release as a user-designated number of beats per minute. Likewise, the option 1412 may be selected to use temperature as the biometric and may be accompanied by an input box 1418 at which the end-user can enter numerical input to establish the threshold/trigger for scent release as a user-designated temperature in Fahrenheit (or Celsius). The option 1414 may be selected to use breath rate as the biometric and may be accompanied by an input box 1420 at which the end-user can enter numerical input to establish the threshold/trigger for scent release as a user-designated number of breaths per minute. Additionally, note that the GUI 1400 may include another option 1422 to select a certain point or event within content presentation to use as a trigger for scent release as well.

If desired, the GUI 1400 may even include a setting 1424 at which the user may establish a particular amount of scent to be released responsive to a trigger. The user may do so by directing numerical input to input box 1426 for example, where the numerical input establishes a scent amount of a particular number of micrograms per meter cubed. Still further, in some examples the GUI 1400 may also include a selector 1428 to initiate a pairing process where one or more of the masks, wearable, and/or smartphone (or other device) may be paired with one another for Bluetooth or other wireless communication to operate consistent with present principles.

Moving on from FIG. 14, note that present principles may apply not just to masks but also to other head-mounted devices (HMDs) with corresponding scent-release electronic assemblies like the assembly 306. For example, smart glasses, an augmented reality (AR) headset, mixed reality (MR) headset, virtual reality (VR) headset, or another type of HMD might include an electronic assembly like the assembly 306 to release scent in the vicinity of the user's nose (e.g., even if the nose is not covered by a facial covering). Biometric sensors for sensing biometrics as discussed above might also be disposed on those HMDs just as they might on a mask. Further note that biometric sensors on a smart phone or other coordinating device may also be used in certain situations consistent with present principles.

Also note consistent with present principles that in some examples, a vibrator on the mask/HMD, other wearable device, or smart phone may even be actuated responsive to each instance of scent release. This may be done to provide a haptic notification to the user that the scent is being released so that, for example, the user does not confuse the released scent with an ambient scent from elsewhere in the user's environment.

Further note that in some examples, scent cartridges may include various base or primary scents that can be dynamically mixed and matched with scents from other cartridges to produce a desired scent. For example, a relational database indicating various primary scent amounts to release from each cartridge at the same time to create a desired hybrid scent may be accessed to determine scent amounts to then actually release.

It may now be appreciated that present principles provide for an improved computer-based user interface that increases the functionality and ease of use of the devices disclosed herein. The disclosed concepts are rooted in computer technology for computers to carry out their functions.

It is to be understood that whilst present principals have been described with reference to some example embodiments, these are not intended to be limiting, and that various alternative arrangements may be used to implement the subject matter claimed herein. Components included in one embodiment can be used in other embodiments in any appropriate combination. For example, any of the various components described herein and/or depicted in the Figures may be combined, interchanged, or excluded from other embodiments.

What is claimed is:

1. At least one mask device, comprising:
a facial covering;
at least one processor;
an electronic assembly accessible to the at least one processor; and
storage accessible to the at least one processor and comprising instructions executable by the at least one processor to:
identify a trigger based on a biometric of a user; and
based on the identification of the trigger, release a scent from the electronic assembly into a space between the facial covering and an area where the user's nose is disposed while wearing the mask.

2. The mask device of claim 1, wherein the trigger comprises the user's heart rate surpassing a threshold heart rate.

3. The mask device of claim 1, wherein the trigger comprises a body temperature of the user surpassing a threshold temperature.

4. The mask device of claim 1, wherein the trigger comprises the user's breathing rate surpassing a threshold breathing rate.

5. The mask device of claim 1, wherein the electronic assembly comprises one or more chambers into which respective scent cartridges are insertable, the scent cartridges housing respective substances having respective scents releasable from the electronic assembly based on identification of the trigger.

6. The mask device of claim 5, comprising the respective scent cartridges.

7. The mask device of claim 5, wherein the chambers provide for an interference fit and/or snap fit of the scent cartridges into the electronic assembly.

8. The mask device of claim 7, wherein the scent cartridges are interchangeable by an end-user via removal from and insertion into the chambers.

9. The mask device of claim 1, wherein the mask comprises one or more vents through which the scent is able to travel from the electronic assembly into the space.

10. The mask device of claim 1, wherein the at least one mask device comprises a mask bearing the electronic assembly and the facial covering and also comprises another device that wirelessly communicates with the mask to release the scent.

11. A method, comprising:
identifying a trigger for releasing a scent via an electronic assembly engaged with a mask; and
based on identifying the trigger, actuating the electronic assembly engaged with the mask to release the scent into a space between a facial covering of the mask and an area where a user's nose is disposed while wearing the mask.

12. The method of claim 11, comprising:
identifying the trigger based on a biometric of the user.

13. The method of claim 12, wherein the biometric relates to a current heart rate of the user.

14. The method of claim 12, wherein the biometric relates to a current body temperature of the user.

15. The method of claim 11, comprising:
identifying the trigger based on execution of a video game.

16. The method of claim 11, comprising:
identifying the trigger based on presentation of an audio video (A/V) program.

17. The method of claim 16, wherein A/V program comprises one or more of: a movie, a television show.

18. The method of claim 11, comprising:
identifying the trigger based on presentation of music or other audio content.

19. A scent cartridge receptacle, comprising:
a housing engageable with a face mask and/or other head-mountable device;
one or more chambers in the housing into which respective scent cartridges are receivable; and
electronic circuitry for communicating with at least one processor to release scent from one or more of the respective scent cartridges via the mask and/or other head-mountable device.

20. The scent cartridge receptacle of claim 19, comprising the at least one processor.
